(19) United States
(12) Patent Application Publication
(10) Pub. No.: US 2003/0073875 A1
(43) Pub. Date: Apr. 17, 2003

(54) Process for the conversion of mixed C4 and C5 streams to motor fuel

(75) Inventors: J. Yong Ryu, Pasadena, TX (US); Lawrence A. Smith JR., Pasadena, TX (US); Abraham P. Gelbein, Falls Church, VA (US); Frits Dautzenberg, Mahwah, NJ (US)

Correspondence Address: KENNETH H. JOHNSON, P.O. BOX 630708, HOUSTON, TX, 77263, US

(73) Assignee: CATALYTIC DISTILLATION TECHNOLOGIES

(21) Appl. No.: 10/071,341

(22) Filed: Feb. 8, 2002

Related U.S. Application Data
(60) Provisional application No. 60/329,451, filed on Oct. 15, 2001.

(51) Int. Cl. C07C 2/00; C07C 2/58

(52) U.S. Cl. 585/331; 585/332

(57) ABSTRACT

An integrated process for upgrading a mixed C₄ or mixed C₅ alkane/isoalkane stream first separates the iso from the normal alkane by distillation. Then a portion of the normal alkane is dehydrogenated to the corresponding alkene. The isoalkane and normal alkene are then fed to an alkylation unit where they are reacted together to form a branched chain alkane having a desirable octane number. If desired a portion of the separated normal alkane may be skeletally isomerized to additional isoalkane.

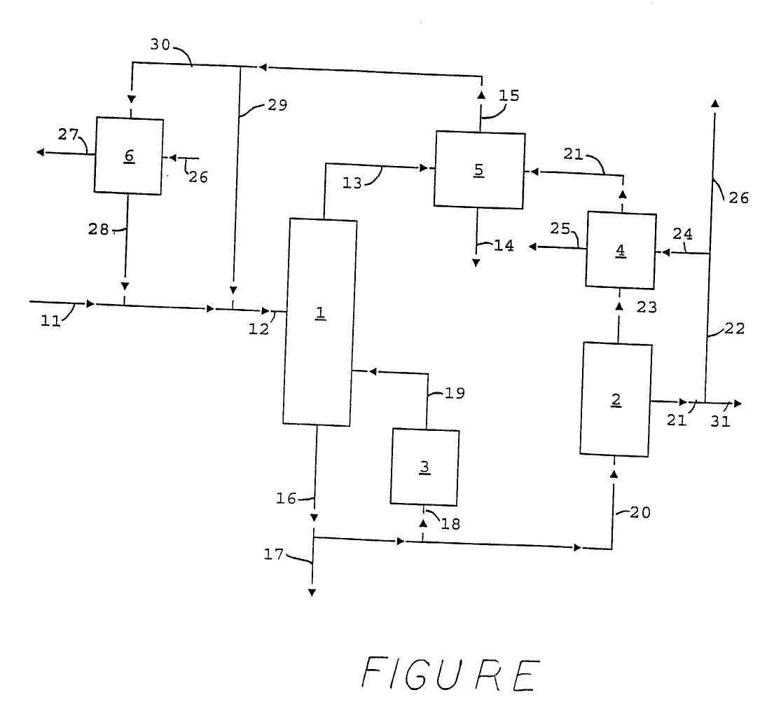

### BACKGROUND OF THE INVENTION

1. Field of the Invention

The present invention relates to an integrated process for upgrading C₄ and C₅ alkanes to motor gasoline. More particularly the invention relates to a process having steps wherein the normal alkanes are separated from the isoalkanes, normal alkanes are dehydrogenated to normal alkenes and isomerized to isoalkanes and the isoalkanes alkylated with the normal alkanes.

Related Information

Mixed C₄-C₅ alkanes have been converted to motor gasoline blending components in the past by several methods. Probably the most common is by use of the isoalkanes in the cold acid alkylation of normal olefins to produce C₈-C₁₀ branched alkanes which are high in octane number. The isoalkanes may also be dehydrogenated and used along with other isoolefins in the production of tertiary ethers (MTBE and TAME). The isoolefins may also be dimerized and then hydrogenated to produce high octane components.

### SUMMARY OF THE INVENTION

In brief the present invention comprises an integrated process for the conversion of mixed C₄ or mixed C₅ alkane streams to produce motor gasoline. The mixed alkane stream is first subjected to separation of the isoalkane from the normal alkane as by fractional distillation. The isoalkane is fed directly to an alkylation unit where the isoalkane is reacted with a normal olefin to produce a branched paraffin. A portion of the normal alkane may be subjected to isomerization to isoalkane which is fed to the alkylation unit. From the remainder of the normal alkane a portion is dehydrogenated to normal olefin which is then used as the olefin feed for the alkylation.

### BRIEF DESCRIPTION OF THE DRAWING

The FIGURE is a simplified flow diagram of one embodiment of the invention.

### DETAILED DESCRIPTION OF THE PREFERRED EMBODIMENT

Referring now to the FIGURE there is shown a flow diagram in schematic form of one embodiment of the invention. The normal/isoalkane mixture (either a C₄ or C₅) is fed via line 11 to a separator 1 where the isoalkane is removed via flow line 13 to be fed directly to an alkylation unit for reaction with a normal olefin to produce a branched alkane having a high octane number. Normal alkane from the bottom of the separator 1 is removed via flow line 16. A small portion of this stream is purged via flow line 17 to remove heavy material that may build up in the system. Depending on the composition of the mixed alkane feed stream, a portion of the normal alkane from the bottom is fed to a skeletal isomerization unit 3 via flow line 18 to produce additional isoalkane. The product from the isomerization unit 3, which comprises iso and normal alkane is introduced back to the separator 1 via flow line 19.

The rest of the normal alkane is fed to a dehydrogenation unit 2 through flow line 20 to produce normal olefins and hydrogen. The product from the dehydrogenation unit 2, which comprises normal olefins and alkanes, is fed to a selective hydrogenation unit 4 via flow line 23 to remove very small amounts of dienes normally present in such a product stream. The hydrogen produced is removed via flow line 21 with a small slip stream taken via flow line 31. A portion of the remaining hydrogen is used in the selective hydrogen unit 4 and is fed via flow line 24. Unneeded hydrogen is removed via flow line 26. Unreacted hydrogen is removed from the selective hydrogenation unit 4 via flow line 25. The selectively hydrogenated product is fed to the alkylation unit 5 via flow line 27 where the normal olefin is reacted with isoalkane to produce the desired alkylate product which is removed via flow line 14. Unreacted isoalkane and unreacted olefin are removed via flow line 15. If the olefin content is low enough the effluent in flow line 15 may be fed directly back to separator 1 via flow lines 29 and 12. However, if the olefin content is too high, the effluent may be fed to hydrogenation unit 6 where the olefins are hydrogenated prior to recycling back to the separator 1.

The preferred alkylation process comprises alkylation of isoparaffin with olefin comprising contacting a fluid system comprising acid catalyst, isoparaffin and olefin in concurrent flow, preferably downflow into contact in a reaction zone with internal packing, such as, a coalescer under conditions of temperature and pressure to react said isoparaffin and said olefin to produce an alkylate product. Preferably, the fluid system comprises a liquid and is maintained at about its boiling point in the reaction zone.

The reaction may be carried out in an apparatus comprising a vertical reactor containing a coalescer in the reaction zone, which may comprise the entire column or a portion thereof.

The process is more completely described in co-owned patent application having docket number CDT 1769/79 (U.S. Ser. No. 60/323,227 filed Sep. 14, 2001) which is hereby incorporated by reference.

The preferred alkylation process employs a downflow reactor packed with contacting internals or packing material (which may be inert or catalytic) through which passes a concurrent multi phase mixture of sulfuric acid, hydrocarbon solvent and reactants at the boiling point of the system. Adjusting the pressure and hydrocarbon composition controls the boiling point temperature. The reactor is preferentially operated vapor continuous but may also be operated liquid continuous. The pressure is preferentially higher at the top of the reactor than at the bottom. Adjusting the flow rates and the degree of vaporization controls the pressure drop across the reactor. Multiple injection of olefin is preferred. The product mixture before fractionation is the preferred circulating solvent. The acid emulsion separates rapidly from the hydrocarbon liquid and is normally recycled with only a few minutes residence time in the bottom phase separator. Because the products are in essence extracted from the acid emulsion, the reaction and/or emulsion promoters may be added without the usual concern for breaking the emulsion. The process may be described as being hydrocarbon continuous as opposed to acid continuous.

The coalescer comprises a conventional liquid-liquid coalescer of a type which is operative for coalescing vaporized liquids. These are commonly known as “mist eliminators” or “demisters”. A suitable coalescer comprises a mesh such as a co-knit wire and fiberglass mesh. For example, it has been found that a 90 needle tubular co-knit mesh of wire and fiberglass such as manufactured by ACS Industries LLC of Houston, Tex., can be effectively utilized, however, it will be understood that various other materials such as co-knit wire and teflon (Dupont TM), steel wool, polypropylene, PVDF, polyester or various other co-knit materials can also be effectively utilized in the apparatus.

The invention claimed is:

1. A process for upgrading a C₄, C₅ or mixed stream thereof comprising normal alkane and isoalkane to motor fuel comprising the steps of:
(a) separating the isoalkane from the normal alkane;
(b) subjecting a portion of the separated normal alkane to dehydrogenation in a dehydrogenation unit to produce a normal alkene; and
(c) feed the separated isoalkane and the normal alkene to an alkylation unit where the isoalkane is reacted with the normal alkene to form a branched alkane.

2. The process according to claim 1 wherein a portion of the separated normal alkane is subjected to skeletal isomerization to produce more isoalkane.

3. The process according to claim 1 wherein the effluent from the dehydrogenation is selectively hydrogenation under conditions to remove dienes.

4. An integrated process for upgrading a C₄, C₅ or mixed stream thereof comprising normal alkane and isoalkane to motor fuel comprising the steps of:
(a) separating isoalkane from normal alkane;
(b) isomerizing a portion of the separated normal alkane to isoalkane;
(c) dehydrogenating a portion of the separated normal alkane to produce normal alkene; and
(d) reacting the isoalkane and normal alkene under alkylating conditions to product isoalkane.

5. The process according to claim 4 wherein said separating is by fractional distillation.

6. The process according to claim 5 wherein said normal alkene from (c) is contacted with hydrogen under conditions to selectively hydrogenate dienes.

7. A process for the production of isooctane from a mixed C₄ alkane stream comprising the steps of:
(a) separating isobutane from normal butane;
(b) isomerizing a portion of the separated normal butane to isobutane;
(c) dehydrogenating a portion of the separated normal butane to produce normal butenes;
(d) selectively hydrogenating normal butenes from (c) under conditions to hydrogenate dienes; and
(e) reacting the isobutane and normal butenes under alkylating conditions to product isooctane.

8. A process for the production of isodecane from a mixed C₅ alkane stream comprising the steps of:
(a) separating isopentane from normal pentane;
(b) isomerizing a portion of the separated normal pentane to skeletal isomerization;
(c) dehydrogenating a portion of the remainder of the separated normal pentane to produce normal pentenes;
(d) selectively hydrogenating normal pentenes from (c) under conditions selectively hydrogenate dienes; and
(e) reacting the isopentane with the normal pentenes under alkylating conditions to product isodecane.
